(19) United States
(12) Patent Application Publication
(10) Pub. No.: US 2012/0083677 A1
(43) Pub. Date: Apr. 5, 2012

(54) BLOOD ANALYZER

(75) Inventors: Yoshiharu Koda, Tokyo (JP); Kojiro Koda, Tokyo (JP)

(73) Assignee: Coden Co., Ltd., Tokyo (JP)

(21) Appl. No.: 13/241,317

(22) Filed: Sep. 23, 2011

(30) Foreign Application Priority Data
Oct. 1, 2010 (JP) 2010-223915

(51) Int. Cl. A61B 5/145

(52) U.S. Cl. 600/309

(57) ABSTRACT

A blood analyzer capable of obtaining a blood analysis result instantaneously without sampling blood but rather applying a DC current to blood flowing through an artery and irradiating it with an ultrasonic wave is provided. It is equipped with a main measurement unit 110 having a positive electrode and a negative electrode placed apart from each other, ultrasonic wave transmitting/receiving probes, which are sandwiched between the positive electrode and the negative electrode and placed adjacent to the positive electrode and negative electrode, and ultrasonic wave transmitting probes placed sandwiched between the ultrasonic wave transmitting/receiving probes, each of which is protruding from the surface and, provided inside of the main measurement unit 110, a measuring control unit comprising: a DC application unit, a positive electrode and a negative electrode, an ultrasonic wave output unit, an ultrasonic wave reception unit, and a component analysis.

### CROSS REFERENCE TO RELATED APPLICATIONS

The present application is related to, and claims priority from, Japanese patent application no. 2010-223915 filed Oct. 1, 2010, the entire contents of which is hereby incorporated by reference in its entirety.

### BACKGROUND OF THE INVENTION

1. Field of the Invention

The present invention relates to the blood analyzer, in particular, a blood analyzer capable of obtaining a blood analysis result instantaneously without sampling blood but rather applying a DC current to blood flowing through an artery and irradiating it with an ultrasonic wave.

2. Description of the Prior Arts

The analysis of blood components has historically been done by collecting a blood sample from an artery and analyzing the blood sample in a blood analyzer for professional use. Also available recently is an apparatus capable of analyzing a blood sample collected at home as disclosed in Unexamined Japanese Patent Application 2009-143.

However, these apparatuses of prior art require blood samples for analyzing blood and are not capable of analyzing blood in a non-invasive manner.

Moreover, the problem with the blood analysis according to the prior art is that it takes time between the time of blood sampling and the time of obtaining the analysis result, as it is necessary for a hospital or clinic to send out blood samples to an laboratory or institution which conducts blood analysis on contracts.

### BACKGROUND OF THE INVENTION

The present invention was made to solve such a problem associated with the prior art, and intends to provide a blood analyzer capable of obtaining the analysis result of blood instantaneously without sampling blood but rather applying a DC current to blood flowing through an artery and irradiating it with an ultrasonic wave.

The blood analyzer according to the present invention is equipped with a positive electrode, a negative electrode, ultrasonic wave transmitting/receiving probes, ultrasonic wave transmitting probes, a DC voltage application unit, an ultrasonic wave output unit, an ultrasonic wave reception unit, and a component analysis unit.

The positive electrode and the negative electrode are placed separately on a blood vessel where the measurement can be made easily. The ultrasonic wave transmitting/receiving probes are placed above a blood vessel adjacent and close to either the positive electrode or the negative electrode. The ultrasonic wave transmitting probes are placed above the blood vessel between the positive electrode and the negative electrode in order to agitate the blood inside the vessel. The DC voltage application unit applies a DC voltage on the positive electrode and the negative electrode. The ultrasonic wave output unit emits the ultrasonic wave from the ultrasonic wave transmitting/receiving probes. The ultrasonic wave reception unit receives the reflecting wave from the blood via the ultrasonic wave transmitting/receiving probes. The component analysis unit operates the DC voltage application unit, the ultrasonic wave output unit and the ultrasonic wave reception unit, and analyzes the blood components based on the waveform of the reflected wave received by the ultrasonic wave reception unit.

The blood analyzer according to the present invention is capable of obtaining the analysis result of blood instantaneously as it analyzes blood without sampling the blood but rather applying a DC current to the blood flowing through an artery and irradiating it with an ultrasonic wave.

These and other features of the invention will be more fully understood by reference to the following drawings.

### BRIEF DESCRIPTION OF THE DRAWINGS

FIG. 1 is an external view of a blood analyzer according to the present invention.

FIG. 2 are detail views of the main measurement unit of the blood analyzer according to the present invention. FIG. 2(a) is a detail view of the outer surface (outside) of the main measurement unit, while FIG. 2(b) is a detail view of the contact surface (inside) of the main measurement unit that contacts a wrist.

FIG. 3 is a diagram showing the locations of arteries in the wrist of a subject being tested.

FIG. 4 is a block diagram of the control system of the blood analyzer according to the present invention.

FIG. 5 is a diagram showing schematically the positional relations ships between the electrodes, probes and blood veins when the blood analyzer according to the present invention is mounted on the wrist.

FIG. 6 is a main flowchart representing the measuring operations of the blood analyzer according to the present invention.

FIG. 7 is a flowchart representing the measuring operations of the blood analyzer according to the present invention.

FIG. 8 is a flowchart representing the measuring operations of the blood analyzer according to the present invention.

FIG. 9 is a flowchart representing the measuring operations of the blood analyzer according to the present invention.

FIG. 10 is a diagram showing transmitting and receiving waveforms during the measurement.

### DETAILED DESCRIPTION OF THE INVENTION

The blood analyzer according to the present invention is described in detail with reference to the accompanying drawings.

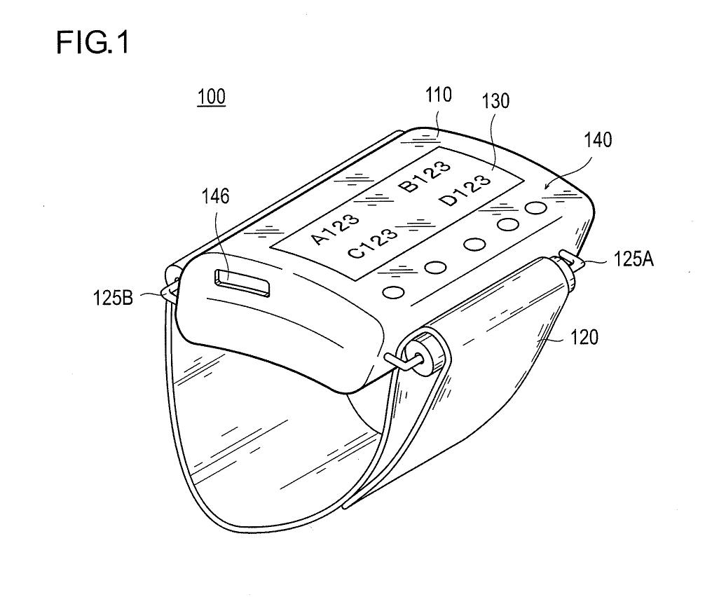

FIG. 1 shows an external view of a blood analyzer according to the present invention. As shown in the drawing, the blood analyzer 100 consists of a measuring main measurement unit 110, and a band 120 for affixing the main measurement unit 110 to a person's body. The band 120 is latched on band affixing parts 125A and 125B provided on the opposing sides of the main measurement unit 110. The main measurement unit 110 is, for example, affixed to the wrist with the band 120.

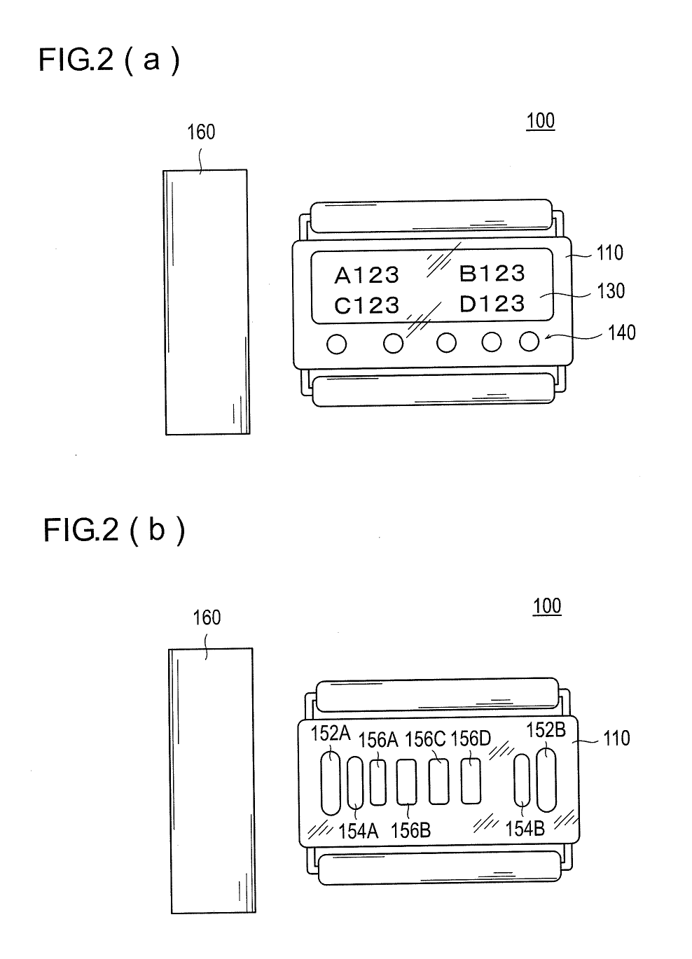

On the outer surface of the main measurement unit 110 provides a display unit 130 displaying the analysis result of the blood and a control button pad 140 consisting of a variety of control buttons. On the contact surface of the wrist unit of the main measurement unit 110 provides the electrodes used for analyzing the blood and the probes (refer to FIG. 2(b)). On the side of the main measurement unit 110 further provides a connection terminal (USB connector) 146 for outputting the analysis result data obtained from the blood.

FIG. 2 shows detail views of the main measurement unit 110 of the blood analyzer 100 according to the present invention. FIG. 2(a) shows the detail of the outer surface of the main measurement unit 110, while FIG. 2(b) shows the detail of the contact surface of the main measurement unit 110 that contacts the patient's wrist. Further, provided on the left side of the main measurement unit 110 is a hemostasis cuff 160 to be wrapped around the wrist to stop bleeding.

Provided on the outer surface of the main measurement unit 110 is the display unit 130 and the control button pad 140 shown below the display unit 130 in the drawing. Displayed on the display unit 130 is the analysis result of the blood components, more specifically the analysis result of the blood of the person examined, as well as the analysis results of an unspecified number of healthy subjects in numerical values. The subject being tested can ascertain whether the content of a specific blood component is normal or abnormal from the display of the analysis result displayed on the display 130. On the control button pad 140 provides a plurality of control buttons that output an order necessary to analyze the blood components.

On the contact surface of the wrist unit of the main measurement unit 110 provides two electrodes, two ultrasonic wave transmitting/receiving probes, and four ultrasonic wave transmitting probes. Shown in the drawing from left to right are a positive electrode 152A, an ultrasonic wave transmitting/receiving probe 154A, four ultrasonic wave transmitting probes 156A-156D, an ultrasonic wave transmitting/receiving probe 154B, and a negative electrode 152B. The four ultrasonic wave transmitting probes 156A-156D are located between the two ultrasonic wave transmitting/receiving probes 154A and 154B. The four ultrasonic wave transmitting probes 156A-156D and the two ultrasonic wave transmitting/receiving probes 154A and 154B are located between the two electrodes 152A and 152B that are placed 30 mm apart from each other.

The positive electrode 152A, the ultrasonic wave transmitting/receiving probe 154A, the four ultrasonic wave transmitting probes 156A-156D, the ultrasonic wave transmitting/receiving probe 154B, and the negative electrode 152B protrusively provided on the contact surface of the main measurement unit 110 are configured in such a manner as to be energized in the push out direction but free to be pushed back. With such a configuration, these electrodes and probes can conform to a curvy surface of the skin closely when the main measurement unit 110 is mounted on the wrist unit so that the blood component analysis can be made accurately.

Having a close contact of the positive electrode 152A and the negative electrode 152B with the skin, the voltage of the positive electrode 152A and the negative electrode 152B can be effectively applied to the blood. Moreover, by having the ultrasonic wave transmitting/receiving probe 154A, the four ultrasonic wave transmitting probes 156A-156D, the ultrasonic wave transmitting/receiving probe 154B closely contacting the skin, the ultrasonic wave can be effectively applied to the blood so that the accuracy of the blood analysis can be improved. It is also possible to apply gel liquid to the mounting area of the main measurement unit 110 in order to cause these electrodes and probes to make a closer contact with the skin more efficiently.

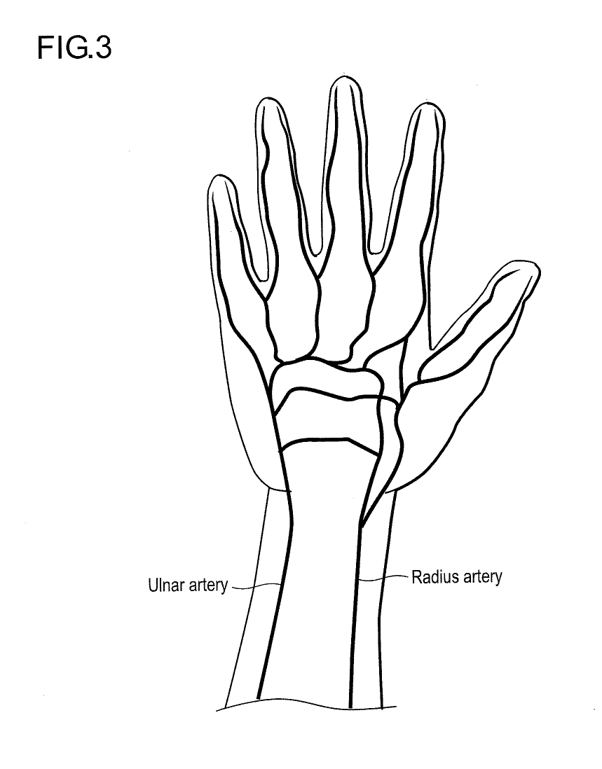

FIG. 3 is a diagram showing the locations of arteries in the wrist of a subject being tested.

As shown in the drawing, there are two major arteries, the ulnar artery and radius artery, in the wrist area of a human being on which is mounted the blood analyzer 100 according to the present invention.

As described in the above, the contact surface of the wrist area of the main measurement unit 110 is provided with an unspecified number of electrodes and probes. In mounting the main measurement unit 110 on the wrist area, it is necessary to adjust the position of the main measurement unit 110 in such a way as to have these electrodes and probes located above the blood vessel of the ulnar artery or radius artery. It is so designed that the measurement by the blood analyzer 100 would not be started if the mounting position of the main measurement unit 110 is not appropriate (if the electrodes and probes are not located above the blood vessel of the ulnar artery or radius artery), as that would result in an inaccurate blood component analysis result. Although it is not shown, the contact surface of the main measurement unit 110 in contact with the wrist surface is configured in such a manner as to be able to rotate freely within a certain angle relative to the outer surface of the main measurement unit 110. Therefore, the mounting position of the main measurement unit 110 can be adjusted by rotating the contract surface.

A DC voltage is applied between the aforementioned positive electrode 152A and the negative electrode 152B. Its purpose is to attract specific particles and molecules in blood to the positive electrode 152A or the negative electrode 152B. The ultrasonic wave transmitting/receiving probes 154A and 154B irradiates blood with ultrasonic wave and receive ultrasonic wave reflected by the blood. The blood components can be analyzed by analyzing the waveforms of the reflected wave in detail. The ultrasonic wave transmitting probes 156A-156D irradiate the dwelling blood between the ultrasonic wave transmitting/receiving probes 154A and 154B with ultrasonic wave to cause vibrations of the blood to activate the particles and molecules in the blood. When the blood is activated by vibrations, specific components in the dwelling blood with a high viscosity become easier to gather around the positive electrode 152A or the negative electrode 152B.

In mounting the main measurement unit 110 on the wrist area of the subject being tested, the hemostasis cuff 160 as shown in FIG. 2 is mounted on the heart side (closer to the shoulder) of the main measurement unit 110 and stop the blood flow temporarily by pumping air to the hemostasis cuff 160.

As described above, the hemostasis cuff 160 is preferably provided as a separate entity from the main measurement unit 110. However, it is also possible to cause the band 120 of the main measurement unit 110 to serve as a hemostasis cuff for the convenience sake to arrest hemorrhage with the band 120.

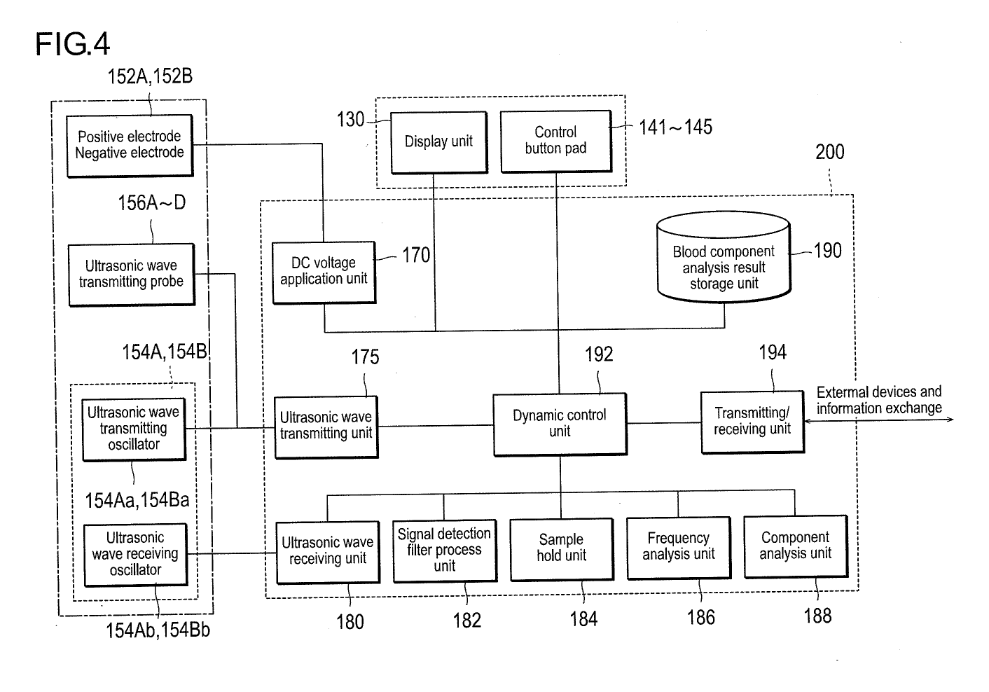

FIG. 4 is a block diagram of the control system of the blood analyzer 100 according to the present invention. A measuring control unit 200 that constitutes the control system of the blood analyzer 100 is built into the main measurement unit 110 and consists of a one-chip microcomputer.

The positive electrode 152A, the negative electrode 152B, the ultrasonic wave transmitting/receiving probes 154A and 154B, and the ultrasonic wave transmitting probes 156A-156D shown in FIG. 4 are located on the contact surface of the main measurement unit 110 that is in contact with the wrist area as shown in FIG. 2 (b). When the main measurement unit 110 is mounted on the wrist area, the positive electrode 152A and the negative electrode 152B are located above the blood vessel 30 mm apart from each other. The ultrasonic wave transmitting/receiving probes 154A and 154B are located above the blood vessel sandwiched between the positive electrode 152A and the negative electrode 152B and adjacent to them. The ultrasonic wave transmitting/receiving probes 154A and 154B each has ultrasonic wave transmitting oscillators 154Aa and 154Ba respectively, and the ultrasonic wave transmitting oscillators 154Aa and 154Ba emit ultrasonic wave toward the blood. Moreover, the ultrasonic wave transmitting/receiving probes 154A and 154B each has ultrasonic wave oscillators 154Ab and 154Bb respectively, and the ultrasonic wave oscillators 154Ab and 154Bb receive the ultrasonic wave reflected by the blood and blood vessel walls to output signals with waveforms that correspond to the levels of the received ultrasonic wave.

The ultrasonic wave transmitting/receiving probes 154A and 154B can efficiently collect the specified molecules and particles contained in the blood that are attracted to the positive electrode 152A and the negative electrode 152B, as the probes are located in the vicinities of the positive electrode 152A and the negative electrode 152B.

The ultrasonic wave transmitting probes 156A-156D are located above the blood vessel between the positive electrode 152A and the negative electrode 152B and radiates an ultrasonic wave of a large amplitude having an amplitude of 1.4 mm and a frequency of 12 kHz. Although the dwelling blood has a high viscosity and a slow migration speed, the application of vibration to the blood which is dwelling due to the hemorrhage arrest activates and promotes the migration of the molecules and particles of the blood, thus shortening their collection around the positive electrode 152A and the negative electrode 152B. The ultrasonic wave receiving probes 156A-156D are also sandwiched between the two ultrasonic wave transmitting/receiving probes 154A and 154B.

The display unit 130 is placed on the outer surface of the main measurement unit 110 as shown in FIG. 2(a). Control buttons 141-145 are placed on the control button pad 140.

The measuring control unit 200 is provided inside the main measurement unit 110. The measuring control unit 200 consists of a DC voltage application unit 170, an ultrasonic wave output unit 175, an ultrasonic wave reception unit 180, a signal detection filter process unit 182, a sample hold lower unit 184, a frequency analysis unit 186, a component analysis unit 188, a blood component analysis storage unit 190, a dynamic control unit 192, and a transmitting/reception unit 194.

The DC voltage application unit 170 applies a DC voltage on the positive electrode 152A and the negative electrode 152B. More specifically, the DC voltage application unit 170 applies a positive voltage on the positive electrode 152A and a negative voltage to the negative electrode 152B. The DC voltage application unit 170 applies a low voltage of approximately 0.8 mV between the positive electrode 152A and the negative electrode 152B to generate a DC current of 0.05 mA between them. The reason for applying a DC voltage here, not an AC voltage, is so that the specific components of blood can be collected on the positive electrode 152A or the negative electrode 152B.

The ultrasonic wave output unit 175 emits ultrasonic wave via the ultrasonic wave transmitting oscillators 154Aa and 154Ba of the ultrasonic wave transmitting/receiving probes 154A and 154B. The ultrasonic wave transmitting oscillators 154Aa and 154Ba irradiate the blood inside the blood vessel with ultrasonic wave. The ultrasonic wave output unit 175 emits ultrasonic wave via the ultrasonic wave transmitting probes 156A-156D. The ultrasonic wave transmitting probes 156A-156D irradiate the blood inside the blood vessel with ultrasonic wave to activate the blood. The ultrasonic wave output unit 175 is capable of modifying the frequency of its ultrasonic wave in correspondence to the type of the particle, which is the subject of the measurement, e.g., blood sugar, cholesterol, etc.

The ultrasonic wave reception unit 180 receives the wave reflected by the blood via the ultrasonic wave receiving oscillators 154Ab and 154Bb the ultrasonic wave transmitting/receiving probes 154A and 154B. The components and their contents of the blood can be finely analyzed by closely analyzing the sizes and shapes of the reflected wave reflected by the blood.

The signal detection filter process unit 182 applies a filter on the reflected waveform of the reflected wave to extract a reflective waveform that corresponds to a component of the blood from the reflected waveform of the reflected wave received by the ultrasonic wave reception unit 180. This is because the characteristic of the reflected waveform varies with each component of the blood.

The sample hold unit 184 holds the reflected waveform that corresponds to a specific component of the blood extracted by the signal detection filter process unit 182. This is because it is necessary to conduct various analyses based on the reflected waveform being held in order to conduct analysis in the latter stage.

The frequency analysis unit 186 is a part for analyzing the frequency component of the reflected waveform of the specific blood component held in the sample hold unit 184. The frequency analysis unit 186 has a circuit having a FFT (fast Fourier transformation) consisting of many groups of filters. It is possible to grasp what kind of a component is contained in the blood by examining what kind of a frequency component is contained with what kind of intensity in the reflected waveform.

The component analysis unit 188 determines the content of each component of the blood based on the characteristics of the reflected waveform analyzed by the frequency analysis unit 186. The component analysis unit 188 stores the characteristics of the reflected waveform corresponding to a component and the content of the particular component of the blood. Thus, it is possible to determine precisely which component is contained in which degree by cross-referencing the feature of the reflected waveform analyzed by the frequency analysis unit 186 with the feature of the reflected waveform stored in the component analysis unit 188. The features of a reflected waveform stored in the component analysis unit 188 concern with that of the reflected waveform after it is processed with a filter by the signal detection filter process unit 182. This is because it is difficult to obtain an accurate analysis result on the components if it is based on the original format of the reflected waveform as it is received by the ultrasonic wave reception unit 180. If we are not so concerned with the accuracy of the analysis, we may conduct a component analysis of the blood based on the characteristics of the original waveform as it is received by the ultrasonic wave reception unit 180.

The blood component analysis storage unit 190 stores the analysis result of the blood components of the subject being tested in a chronological order as well as the analysis results of the bloods of an unspecified number of healthy subjects in a chronological order. The stored analysis results can be displayed on the display unit 130.

The dynamic control unit 192 generally controls the operations of the blood analyzer 100. It controls the operations of the blood analyzer 100 when the start of a measurement or the interruption of a measurement is instructed by the control button buttons 141-145. Also, during a measurement, it controls all the operations sequentially from the start to end of the measurement by issuing instructions to the constituents of the blood analyzer 100 according to the stored measurement program.

The transmitting/reception unit 194 communicates with other blood analyzers 100 and central management equipment (that files the dater of blood analyzers 100 all over the nation) and exchanges analysis results stored in the blood component analysis result storage unit 190 with the central management equipment, for example, via the Internet circuit.

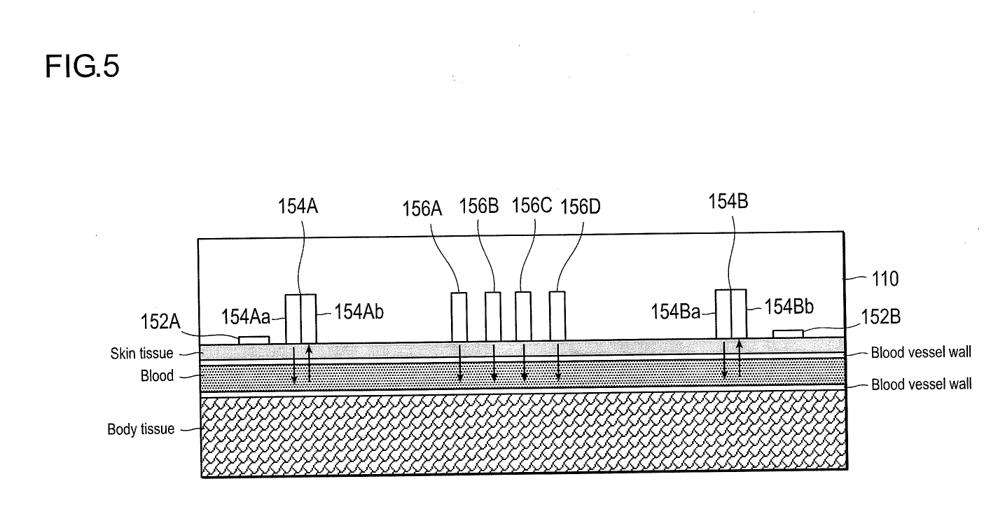

FIG. 5 is a diagram showing schematically the positional relations ships between the electrodes, probes and blood veins when the blood analyzer 100 according to the present invention is mounted on the wrist.

As shown in the drawing from left to right, the positive electrode 152A, the ultrasonic wave transmitting/receiving probe 154A (ultrasonic wave transmitting oscillator 154Aa, ultrasonic wave receiving oscillator 154Ab), four ultrasonic wave transmitting probes 156A-156D, the ultrasonic wave transmitting/receiving probe 154B (ultrasonic wave transmitting oscillator 154Ba, ultrasonic wave receiving oscillator 154Bb), and the negative electrode 152B are contacting the skin tissue of the wrist area. These electrodes and probes are in close contacts with the skin tissue as they are energized by a cushion material provided inside the main measurement unit 110 in the push out direction but free to be pushed back.

Applying a positive voltage to the positive electrode 152A and the negative voltage to the negative electrode 152B causes a weak current to run between the positive electrode 152A and the negative electrode 152B through the skin tissue, blood vessel walls, and blood. The particles and molecules contained in blood plasma include components with minus ions, those with plus ions, and those with no ions. The particles and molecules with minus ions are attracted to the positive electrode 152A, while the particles and molecules with plus ions or no ions are attracted to the negative electrode 152B. While the reason for applying the DC voltage is to collect those charged components to the respective electrodes as described above, it may cause a negative effect on the human body to maintain the application for a long time, so that it is limited to a short period of time, e.g., 40 seconds or so, in this invention. It is dangerous to apply a high DC voltage or an AC voltage as it can affect the pulse due to the electromotive force of the cardiac muscle tissue.

When the blood in a blood vessel is irradiated with ultrasonic wave by the ultrasonic wave transmitting probes 156A-156D, the blood becomes activated and blood components are attracted to the positive electrode 152A and the negative electrode 152B. The blood contains a large amount of various particles and molecules. Some of these particles exist as single granular particles, while others exist in spiral or chain-like short forms. Although the viscosity of blood varies from one person to another, they all have some viscosity. Since the blood dwelling in the blood vessel as a result of hemorrhage arrest has a higher viscosity, it takes some time for the specific particles or molecules to convene to the electrodes. Since it is dangerous to halt the blood flow in the artery for a long time, a vibration effect by is applied to the dwelling blood by means of ultrasonic wave in order to promote the moves of the particles and molecules to the electrodes. Since the particles and molecules in the dwelling blood under the vibration effect perform same as they do when the viscosity is low, the time required for causing the particular particles and molecules to convene to the electrodes can be substantially reduced, thus reducing the measurement time. The time required for causing the molecules to convene to the electrodes differ depending on the kinds of molecules and the electric charges the molecules carry when more closely observed. However, according to experiments, it was confirmed that it is possible to reduce the viscosity of the blood when it is irradiated with ultrasonic wave with a wave length of 1.4 mm and a frequency of 6 kHz from the ultrasonic wave transmitting probes 156A-156D, reducing the measuring time of the blood components from 8 minutes when no ultrasonic wave irradiation is applied to 40 seconds when it is applied.

Moreover, particles of smaller molecular weights and molecules with smaller electric charge take longer times to move from the positive electrode 152A to the negative electrodes 152B compared to those with larger amounts of molecular weights or electric charges. Therefore, the times of f the molecules in blood take to move a distance various according to the DC voltage applied to the positive electrode 152A and the negative electrode 152B as well. Although said motion times can be shortened by applying a higher DC voltage, care need to be taken as an excessively high DC voltage may cause bad effects on the human body.

The ultrasonic wave transmitting/receiving probe 154A irradiates the components and molecules of blood convened around the positive electrode 152A with an ultrasonic wave transmitted from the ultrasonic wave transmitting oscillator 154Aa, and receives the reflected wave via the ultrasonic wave receiving oscillator 154Ab. The ultrasonic wave transmitting/receiving probe 154B irradiates the components and molecules of blood convened around the negative electrode 152B with an ultrasonic wave transmitted from the ultrasonic wave transmitting oscillator 154Ba, and receives the reflected wave via the ultrasonic wave receiving oscillator 154Bb.

In order to analyze the blood components, the ultrasonic wave transmitting/receiving probe 154A and the ultrasonic wave transmitting/receiving probe 154B have to be located correctly above the blood vessel. Whether the ultrasonic wave transmitting/receiving probe 154A and the ultrasonic wave transmitting/receiving probe 154B are correctly located above the blood vessel or not can be judged by checking if the characteristics of the reflected waveforms received by the ultrasonic wave transmitting/receiving probe 154A and the ultrasonic wave transmitting/receiving probe 154B match with the reference characteristics of the reflected waveforms respectively. The reflected waveforms for reference are store in the dynamic control unit 192 shown in FIG. 4. If the characteristics of the reflected waveform received by each of the ultrasonic wave transmitting/receiving probe 154A and the ultrasonic wave transmitting/receiving probe 154B do not match with the characteristics of the reflected waveforms for reference, it means that the ultrasonic wave transmitting/receiving probe 154A and the ultrasonic wave transmitting/receiving probe 154B are not correctly located above the blood vessel, so that the measurement of the blood analyzer 100 does not start.

The configuration of the blood analyzer according to the present invention is as follows.

Blood consists of various particles and molecules residing in blood plasma, including erythrocytes, leukocytes, platelets, lymphocytes, albumin, globulin, protein, carbohydrate, neutral fat, HDL cholesterol, total cholesterol, salt, electrolytes, and LDH.

In order to determine the contents of particles and molecules contained in blood plasma, it has hitherto been necessary to collect a blood sample from a blood vessel using a syringe and numerically determine the particles and molecular weights by a quantitative assay of the blood specimen. This test of prior art caused a pain to the subject being tested, put a limit to the amount of sample to be taken a day, and took some time to obtain the analysis result, so that it was very inconvenient when an emergency treatment is required.

The blood analyzer according to the present embodiment does not require any blood sample, and allows us to obtain the analysis result of the blood components instantaneously with the measurement. Moreover, since there is no physical contact with blood, it prevents infections inside the hospital or building. Consequently, the invention can eliminate all the problems existed before.

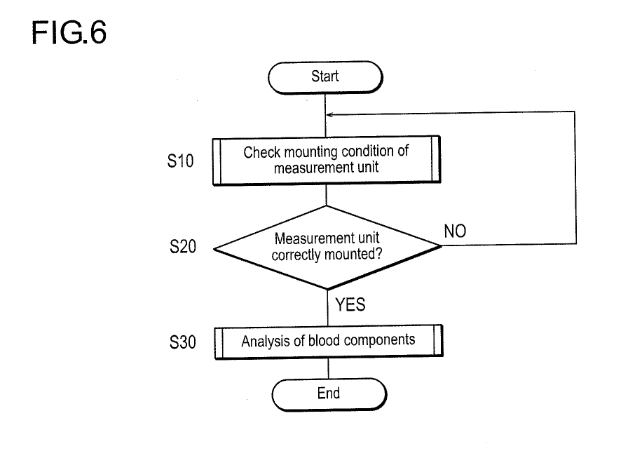

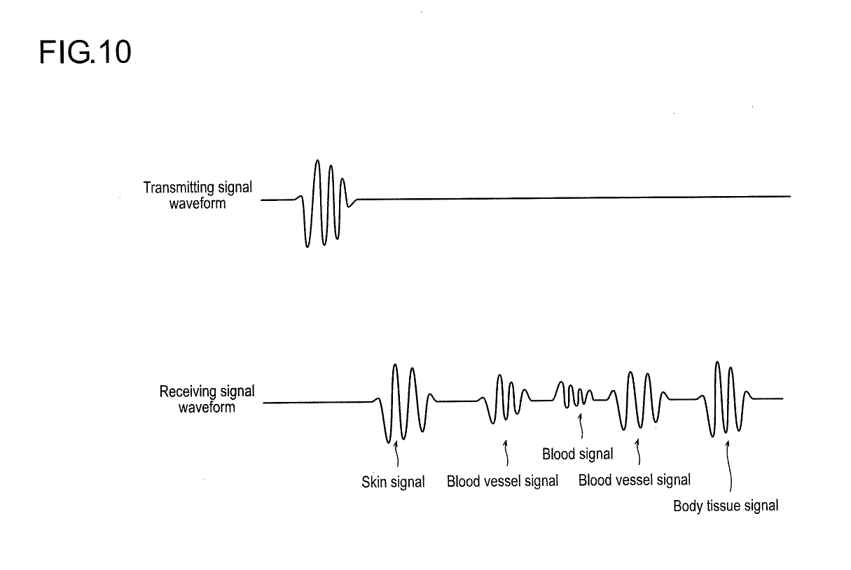

The specific operations of the blood analyzer according to the present invention with reference to the flowcharts of FIG. 6 to FIG. 9, as well as waveform charts of FIG. 10 and FIG. 11.

FIG. 6 is a main flowchart representing the measuring operations of the blood analyzer according to the present invention.

First of all, in executing measurement using the blood analyzer 100 according to the present invention, the main measurement unit 110 is mounted on the wrist area of the subject being tested. The hemostasis cuff 160 is mounted on the heart side (closer to the shoulder) of the main measurement unit 110 and stop the blood flow temporarily by pumping air to the hemostasis cuff 160.

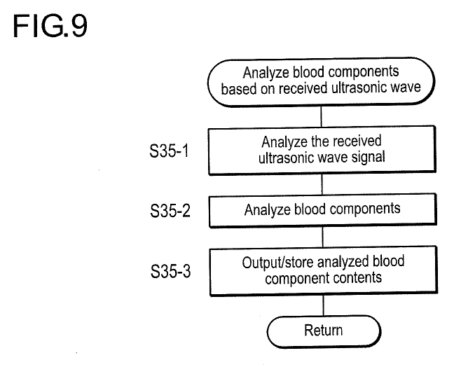

The mounting condition of the main measurement unit 110 is checked at this point. The mounting condition check is done by the blood analyzer 100 itself as the start control button is pressed. As the subroutine flowchart of this process is shown in FIG. 9, the detail description is done below with reference to FIG. 9 (step S10).

Next, if the mounting condition of the main measurement unit 110 is not correct (step S20: No), the system does not advance to the blood component analysis, but rather returns to step S10 to recheck the mounting condition of the measurement unit 110. In this case, an error signal indicating that the mounting condition is not normal is displayed on the display unit 130 of the main measurement unit 110. Noticing this error signal, the subject being tested correct the mounting position of the main measurement unit 110 to locate the electrodes and probes properly above the blood vessel, and then instructs the main measurement unit 110 to check its mounting condition (step S20).

If the mounting condition of the main measurement unit 110 is normal (step S20: Yes), it is the condition that enables an accurate blood analysis, so that the blood analysis by the blood analyzer 100 is initiated. As the subroutine flowchart of this process is shown in FIG. 10, the detail description is done below with reference to FIG. 10 (step S30).

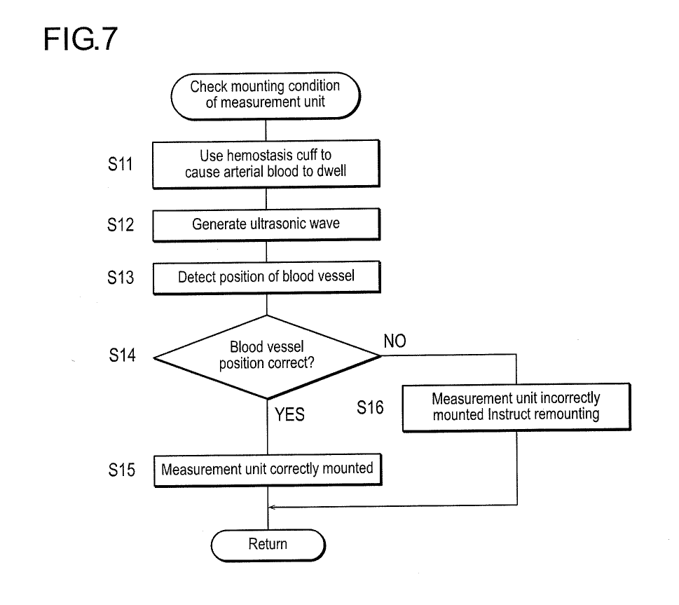

FIG. 7 is a flowchart representing the measuring operations of the blood analyzer according to the present invention. This flowchart is a subroutine flowchart indicating the specific process of S10 shown in FIG. 6.

In checking the mounting condition of the main measurement unit 110, the hemostasis cuff 160 is inflated to stop the arterial blood flow of the wrist area to cause the blood to dwell in the blood vessel as shown above (step S11).

The ultrasonic wave output unit 175 radiates ultrasonic wave from both the ultrasonic wave transmitting oscillator 154Aa and the ultrasonic wave transmitting oscillator 154Ba of the ultrasonic wave transmitting/receiving probe 154A and the ultrasonic wave transmitting/receiving probe 154B towards the blood vessel. The transmitting signal waveform when the ultrasonic wave is radiated is a pulse-like waveform with a constant frequency as shown in the upper waveform of FIG. 10 (step S12).

The ultrasonic wave reception unit 180 receives ultrasonic wave reflected by the blood vessel via both the ultrasonic wave receiving oscillator 154Ab and the ultrasonic wave receiving oscillator 154Bb of the ultrasonic wave transmitting/receiving probe 154A and the ultrasonic wave transmitting/receiving probe 154B towards the blood vessel. The reflected wave contains a skin signal, a blood vessel signal, a blood signal and a body tissue signal in a chronological order as shown in the lower waveform of FIG. 10. A signal detection filter process unit 182 and a sample hold unit 184 extract the portion that corresponds to the blood vessel signal from the reflected waveform formed by the reflected wave, and a dynamic control unit 192 checks if it matches with the reflected waveform for reference. If it is confirmed that a waveform that matches with the blood vessel signal, it means that the blood vessel is located beneath the main measurement unit 110 and the ultrasonic wave transmitting/receiving probe 154A and the ultrasonic wave transmitting/receiving probe 154B are located properly above the blood vessel (step S13).

If the dynamic control unit 192 judges that a waveform identical with that of the blood vessel signal exists, it means that the blood vessel is located properly (step S14: Yes), it is judged that the mounting condition of the main measurement unit 110 is correct (step S15). On the other hand, if the dynamic control unit 192 judges that a waveform identical with that of the blood vessel signal does not exist, it is judged that the blood vessel is not located properly (step S15: No), and that the mounting condition of the main measurement unit 110 is incorrect, so that it is instructed to repeat the mounting of the main measurement unit 110 on the display unit 130 (step S16).

Moreover, although the mounting condition of the main measurement unit 110 is checked after the hemostasis cuff 160 is inflated in said flowchart, it can be configured to make a judgment whether the mounting condition is correct or not by simply mounting the main measurement unit 110 on the wrist area without using the hemostasis cuff 160.

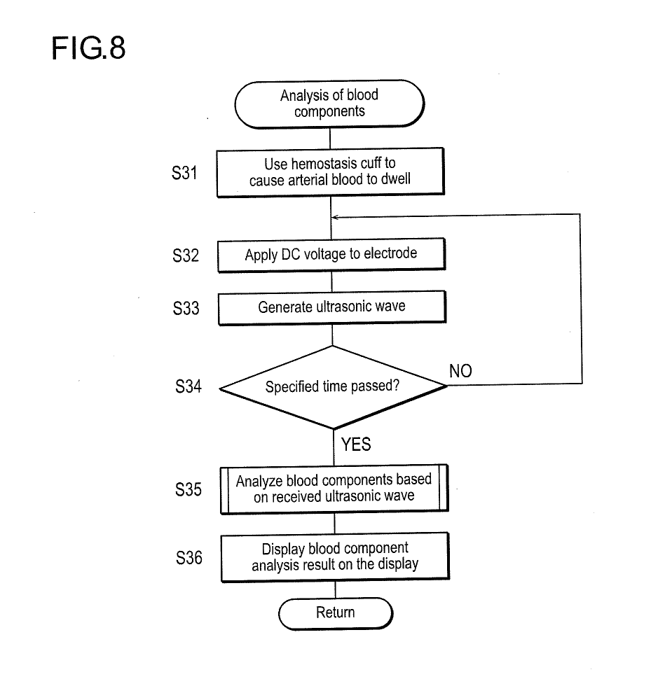

FIG. 8 is a flowchart representing the measuring operations of the blood analyzer according to the present invention. This flowchart is a subroutine flowchart indicating the specific process of S30 shown in FIG. 6.

First, inflate the hemostasis cuff 160 located on the shoulder side of the main measurement unit 110, which is mounted at a proper location in the wrist area, and stop the blood flow in the artery located beneath the main measurement unit 110 to cause the blood to dwell in the blood vessel. Air supply to the hemostasis cuff 160 automatically stops when the air pressure inside the hemostasis cuff 160 reaches 2 atm (atmospheric pressure). When the measurement described below is completed, the air will be vented automatically from the hemostasis cuff 160 to bring back the blood flow (step S31).

Next, the DC voltage application unit 170 applies a DC voltage between the positive electrode 152A and the negative electrode 152B. More specifically, it is a voltage of 0.8 mV (step S32).

Approximately simultaneous with the start of the process of the above step, the ultrasonic wave output unit 175 emits ultrasonic wave via the ultrasonic wave transmitting oscillator 154Aa and the ultrasonic wave transmitting oscillator 154Ba of the ultrasonic wave transmitting/receiving probe 154A and the ultrasonic wave transmitting/receiving oscillator 154B, as well as the ultrasonic wave transmitting probes 156A-156D. The ultrasonic waves thus emitted excite the particles and molecules of the blood dwelling inside the blood vessel (step S33).

The application of the DC voltage in step S22 and the emission of the ultrasonic wave in step S23 are applied simultaneously for a specified time period. For example, in order to quantify the amount of neutral fat contained in the blood, a voltage of 0.8 mV is applied between the positive electrode 152A and the negative electrode 152B by means of the DC voltage application unit 170 to cause a DC current of approximately 0.05 mA. This causes the neutral fat molecules in the blood to move toward the negative electrode 152B. Since it takes time to complete the move of the neutral fat molecules to the negative electrode 152B, the neutral fat molecules are prompted to migrate by irradiating the dwelling blood with ultrasonic wave of a large amplitude of 1.2 mm and a frequency of 12 kHz transmitted from the ultrasonic transmitting probes 156A-156D, thus to minimize the moving time. The neutral fat molecules then convene around the negative electrode 152B in approximately 50 seconds. Therefore, the allowance time for step S34 in measuring neutral fat is set at 50 seconds. Also, the allowance time for step S34 in measuring the total cholesterol amount in blood is set at 45 seconds (step S34).

When said time has passed, the ultrasonic wave output unit 175 emits an ultrasonic wave of a frequency of 2 MHz and a wavelength of 3 mm from the ultrasonic wave transmitting oscillators 154Az and 154Ba. The ultrasonic wave reception unit 180 then analyzes the components contained in the blood, e.g., neutral fat and total cholesterol, based on the reflected wave received via the ultrasonic wave receiving oscillator 154Ab and 154Bb. Although it is configure to have the ultrasonic wave to be emitted from both the ultrasonic wave transmitting oscillators 154Aa and 154Ba in the above example, it is also possible to configure to emit the ultrasonic wave from ultrasonic wave transmitting oscillator 154Ba alone because the neutral fat molecules convene around the negative electrode 152B side. The specific method of this analysis will be described below based on the flowchart of FIG. 9 (step S35).

As mentioned above, the blood contents according to the analysis are displayed on the display unit 130 of the main measurement unit 110 (step S36). Therefore, the subject being tested will be able to know the contents of his/her own blood within only a minute or so from the start of the measurement.

FIG. 9 is a flowchart representing the measuring operations of the blood analyzer according to the present invention. This flowchart is a subroutine flowchart indicating the specific process of S35 shown in FIG. 8.

The ultrasonic wave reception unit 180 receives the reflected wave of the receiving signal waveform as shown in FIG. 10. In other words, it is the reflected wave where the skin signal reflected by the skin, the blood vessel signal reflected by the blood vessel, the blood signal reflected by the components of the blood, the blood vessel signal reflected by the blood vessel, and the body tissue signal reflected by the body tissue are arranged in a chronological order. The signal detection filter process unit 182 extracts the blood signal reflected by the components of the blood by filtering the reflected wave. The ultrasonic wave signal received by the above process is then analyzed.

More specifically, the blood signal that indicates the waveform of the neutral fat is expanded and the expanded signal is amplified logarithmically to adjust the waveform. Furthermore, the waveform is faired up to make the extraction of specific blood components easier by removing waveforms that are unnecessary to detect the neutral fat content and making signals that are going through violent changes chronologically sharper than the basic signals (step S35-1).

Next, the blood component analysis unit 188 analyzes the blood components by referencing the stored waveform and the waveform after the waveform fairing. The component analysis unit 188 stores the characteristics of the reflected waveform corresponding to a component and the content of the particular component of the blood as described above. Thus, it is possible to determine precisely which component is contained in which degree by cross-referencing the feature of the reflected waveform analyzed by the frequency analysis unit 186 with the feature of the reflected waveform stored in the component analysis unit 188 (step S35-2).

Next, the components of the blood thus analyzed are displayed on the display unit 130, and stored in the blood component analysis result storage unit 190. Consequently, as the analysis result of the blood components of the subject being tested gets stored chronologically, the subject being tested can grasp his/her own state of aggravation or recovery of the disease by looking at his/her own past measurement results. Moreover, the analysis result not only gets stored in the blood component analysis result storage unit 190, but also can be stored in an external device via the transmitting/receiving unit 194 (step S35-3).

The measurement of the blood components should be conducted selecting a frequency of a slightly low but sufficient resolution such as 1 MHz for detecting total protein or potassium, whose particle diameter and molecular weight are small, a frequency of approximately 2 MHz for detecting total cholesterol and blood sugar contents, and a frequency of approximately 3 MHz in case a resolution sufficient for detecting smaller particles is desired. As can be seen from the above, low frequency ultrasonic waves result in low resolutions and are suitable for detecting particles of small molecular weights, while high frequency ultrasonic waves result in high resolutions and are suitable for surveying particles and molecules of high molecular weights in detail.

Also, although it is configure in the above embodiment to have a warning to be displayed on the display unit 130 in case the main measurement unit 110 is not mounted properly to prompt remounting without starting the measurement, it can also be configured to prompt by an alarm sound. Although a case of measuring at the wrist in the above embodiment, it goes without saying that a similar measurement can be done at the ankle or any other part of the body.

1. A blood analyzer comprising:
ultrasonic wave transmitting/receiving probes placed above a blood vessel;
ultrasonic wave transmitting probes placed above said blood vessel in order to agitate blood inside said blood vessel;
an ultrasonic wave output unit for emitting an ultrasonic wave from said ultrasonic wave transmitting/receiving probes and said ultrasonic wave transmitting probes;
an ultrasonic wave reception unit for receiving a reflecting wave from said blood via said ultrasonic wave transmitting/receiving probes; and
a component analysis unit for operating said ultrasonic wave output unit and said ultrasonic wave reception unit in order to analyze components of said blood based on the waveform of the reflected wave received by said ultrasonic wave reception unit.

2. A blood analyzer comprising:
a positive electrode and a negative electrode placed apart from each other above a blood vessel;
ultrasonic wave transmitting/receiving probes placed above said blood vessel adjacent to either said positive or negative electrode;
ultrasonic wave transmitting probes placed above said blood vessel between said positive electrode and negative electrode in order to agitate blood inside said blood vessel;
a DC voltage application unit for applying a DC voltage on said positive electrode and negative electrode;
an ultrasonic wave output unit for emitting an ultrasonic wave from said ultrasonic wave transmitting/receiving probes and said ultrasonic wave transmitting probes;
an ultrasonic wave reception unit for receiving a reflecting wave from said blood via said ultrasonic wave transmitting/receiving probes; and
a component analysis unit for operating said DC voltage application unit, said ultrasonic wave output unit and said ultrasonic wave reception unit in order to analyze components of said blood based on the waveform of the reflected wave received by said ultrasonic wave reception unit.

3. A blood analyzer comprising: a positive electrode and a negative electrode placed apart from each other;
ultrasonic wave transmitting/receiving probes sandwiched between said positive electrode and negative electrode being placed close to said positive electrode and negative electrode;
ultrasonic wave transmitting probes placed to be sandwiched between said ultrasonic wave transmitting/receiving probes, and
a main measurement unit protrusively place on a surface, wherein
said main measurement unit having in its inside a measuring control unit comprising:
a DC voltage application unit for applying a DC voltage on said positive electrode and negative electrode;
an ultrasonic wave output unit for emitting an ultrasonic wave from said ultrasonic wave transmitting/receiving probes and said ultrasonic wave transmitting probes;
an ultrasonic wave reception unit for receiving a reflecting wave from said blood via said ultrasonic wave transmitting/receiving probes; and
a component analysis unit for operating said DC voltage application unit, said ultrasonic wave output unit and said ultrasonic wave reception unit in order to analyze components of said blood based on the waveform of the reflected wave received by said ultrasonic wave reception unit.

4. The blood analyzer claimed in claim 3 wherein each of said positive electrode, negative electrode, ultrasonic wave transmitting/receiving probes, and ultrasonic wave transmitting probes provided protrusively on the surface of said main measurement units is configured to be energized in the push out direction but free to be pushed back.

5. The blood analyzer claimed in either one of claims 1 through 3 wherein an unspecified number of said ultrasonic wave transmitting probes are provided.

6. The blood analyzer claimed in claim 3 wherein said main measurement unit is provided with a display unit for displaying a result of the analysis of components of the blood analyzed by said component analysis unit.

7. The blood analyzer claimed in claim 3 wherein said measuring control unit further comprises a blood component analysis result storage unit for storing the analysis result of the components of the blood of a subject being tested in a chronological order as well as the analysis results of the components the bloods of an unspecified number of healthy subjects in a chronological order.

8. The blood analyzer claimed in either one of claims 1 through 3 wherein said blood analyzer starts said measurement only when it can confirm that said ultrasonic wave transmitting/receiving probes are placed above a blood vessel, and does not start the measurement when it cannot confirm that said ultrasonic wave transmitting/receiving probes are placed above a blood vessel,

9. The blood analyzer claimed in either one of claims 1 through 3 wherein said ultrasonic wave transmitting probes excite said blood using an ultrasonic wave with a large amplitude of 1.4 mm and a frequency of 12 kHz.

10. The blood analyzer claimed in either one of claims 2 or 3 wherein said DC voltage application unit applies a low voltage of approximately 0.8 mV between said positive electrode and negative electrode to generate a DC current of 0.05 mA.
